(19) United States
(12) Patent Application Publication
(10) Pub. No.: US 2011/0125264 A1
(43) Pub. Date: May 26, 2011

(54) IMPLANTABLE DEVICES FOR SUBCHONDRAL TREATMENT OF JOINT PAIN

(75) Inventors: Charanpreet S. BAGGA, Basking Ridge, NJ (US); Shaun B. HANSON, West Chester, PA (US); Marc R. VISCOGLIOSI, New Nork, NY (US); Peter F. SHARKEY, Villanova, PA (US)

(73) Assignee: KNEE CREATIONS, LLC, New York, NY (US)

(21) Appl. No.: 12/950,273

(22) Filed: Nov. 19, 2010

Related U.S. Application Data
(60) Provisional application No. 61/263,170, filed on Nov. 20, 2009.
(60) Provisional application No. 61/300,337, filed on Feb. 1, 2010.

(51) Int. Cl. A61F 2/28

(52) U.S. Cl. 623/16.11

(57) ABSTRACT

Devices and associated methods are disclosed for treating bone, and particularly bone tissue at the joints. Disclosed are curved implantable devices that can be used either alone or in combination with this augmentation or hardening material for the repair of bone defects and which are particularly suited for use at the joints, and even more particularly, suited for use at the subchondral bone level.

### CROSS REFERENCE TO RELATED APPLICATIONS

This application claims priority to U.S. Provisional No. 61/300,337 filed Feb. 1, 2010, and entitled “DEVICES AND INSTRUMENTS FOR BONE REPAIR AND METHODS OF USE,” and U.S. Provisional No. 61/263,170 filed Nov. 20, 2009, and entitled “METHOD FOR TREATING JOINT PAIN AND ASSOCIATED INSTRUMENTS,” which are herein incorporated by reference in their entirety.

This application also relates to co-pending and co-owned U.S. patent application Ser. No. ______, filed Nov. 19, 2010 and entitled “SUBCHONDRAL TREATMENT OF JOINT PAIN,” U.S. patent application No. Ser. ______, filed Nov. 19, 2010 and entitled “IMPLANTABLE DEVICES FOR SUBCHONDRAL TREATMENT OF JOINT PAIN,” the contents of which are herein incorporated in their entirety by reference

### FIELD

The present invention relates to devices and instruments for the surgical treatment of bone tissue, and more particularly to devices, instruments and associated methods for the surgical repair and treatment of damaged or compromised bone tissue, especially at or near a joint.

### BACKGROUND

Human joints, in particular the knee, hip and spine, are susceptible to degeneration from disease, trauma, and long-term repetitive use that eventually lead to pain. Knee pain, for example, is the impetus for a wide majority of medical treatments and associated medical costs. The most popular theory arising from the medical community is that knee pain results from bone-on-bone contact or inadequate cartilage cushioning. These conditions are believed to frequently result from the progression of osteoarthritis, which is measured in terms of narrowing of the joint space. Therefore, the severity of osteoarthritis is believed to be an indicator or precursor to joint pain. Most surgeons and medical practitioners thus base their treatments for pain relief on this theory. For example, the typical treatment is to administer pain medication, or more drastically, to perform some type of joint resurfacing or joint replacement surgery.

However, the severity of osteoarthritis, especially in the knee, has been found to correlate poorly with the incidence and magnitude of knee pain. Because of this, surgeons and medical practitioners have struggled to deliver consistent, reliable pain relief to patients especially if preservation of the joint is desired.

Whether by external physical force, disease, or the natural aging process, structural damage to bone can cause injury, trauma, degeneration or erosion of otherwise healthy tissue. The resultant damage can be characterized as a bone defect that can take the form of a fissure, fracture, lesion, edema, tumor, or sclerotic hardening, for example. Particularly in joints, the damage may not be limited to a bone defect, and may also include cartilage loss (especially articular cartilage), tendon damage, and inflammation in the surrounding area.

Patients most often seek treatment because of pain and deterioration of quality of life attributed to the osteoarthritis. The goal of surgical and non-surgical treatments for osteoarthritis is to reduce or eliminate pain and restore joint function. Both non-surgical and surgical treatments are currently available for joint repair.

Non-surgical treatments include weight loss (for the overweight patient), activity modification (low impact exercise), quadriceps strengthening, patellar taping, analgesic and anti-inflammatory medications, and with corticosteroid and/or viscosupplements. Typically, non-surgical treatments, usually involving pharmacological intervention such as the administration of non-steroidal anti-inflammatory drugs or injection of hyaluronic acid-based products, are initially administered to patients experiencing relatively less severe pain or joint complications. However, when non-surgical treatments prove ineffective, or for patients with severe pain or bone injury, surgical intervention is often necessary.

Surgical options include arthroscopic partial meniscectomy and loose body removal. Most surgical treatments conventionally employ mechanical fixation devices such as screws, plates, staples, rods, sutures, and the like are commonly used to repair damaged bone. These fixation devices can be implanted at, or around, the damaged region to stabilize or immobilize the weakened area, in order to promote healing and provide support. Injectable or fillable hardening materials such as bone cements, bone void fillers, or bone substitute materials are also commonly used to stabilize bone defects.

High tibial osteotomy (HTO) or total knee arthroplasty (TKA) is often recommended for patients with severe pain associated with osteoarthritis, especially when other non-invasive options have failed. Both procedures have been shown to be effective in treating knee pain associated with osteoarthritis.

However, patients only elect HTO or TKA with reluctance. Both HTO and TKA are major surgical interventions and may be associated with severe complications. HTO is a painful procedure that may require a long recovery. TKA patients often also report the replaced knee lacks a “natural feel” and have functional limitations. Moreover, both HTO and TKA have limited durability. Accordingly, it would be desirable to provide a medical procedure that addresses the pain associated with osteoarthritis and provides an alternative to a HTO or TKA procedure.

### SUMMARY

The present disclosure provides devices and instruments that can allow precise, controlled injection of an augmentation or hardening material into bone. Also provided are curved implantable devices that can be used either alone or in combination with this augmentation or hardening material for the repair of bone defects and which are particularly suited for use at the joints, and even more particularly suited for use at the subchondral bone level.

In one exemplary embodiment, an implantable device for insertion into a periphery of a bone comprises a curved elongate body extending between a first, leading end and a second, trailing end, the second end including a tool-receiving portion for receiving a tool.

In another embodiment, a method of treating a bone defect near a periphery of the bone, comprises: providing a curved implantable device having a tool-engaging feature; securing the device to an insertion tool; and using the insertion tool, inserting the curved implantable device along the periphery of the bone

In another embodiment, a method of treating a bone defect is provided. The method may comprise the steps of providing a first implantable device, the device having a curved shape forming a concave inner surface, providing a second implantable device, the device having a curved shape forming a concave inner surface, and inserting the first and second implantable devices such that the concave inner surfaces face towards one another and encircle the bone defect.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1A illustrates a perspective view of an exemplary embodiment of an implantable device of the present invention;

FIG. 1B illustrates a perspective view of yet another exemplary embodiment of an implantable device of the present invention;

FIG. 2A illustrates a perspective view of still another exemplary embodiment of an implantable device of the present invention;

FIG. 2B shows the implantable device of FIG. 2A and an insertion tool;

FIG. 2C shows the implantable device and insertion tool of FIG. 2B partially attached;

FIG. 2D shows another perspective view of the implantable device and insertion tool of FIG. 2C;

FIG. 3A illustrates a perspective view of still yet another exemplary embodiment of an implantable device of the present invention;

FIG. 3B illustrates a perspective view of another exemplary embodiment of an implantable device of the present invention;

FIG. 4A illustrates a perspective view of yet another exemplary embodiment of an implantable device of the present invention;

FIG. 4B illustrates a top-down view of the implantable device of FIG. 4A;

FIGS. 4C-4H illustrate a method of using the implantable device of FIG. 4A;

FIG. 5A illustrates a perspective view of still another exemplary embodiment of an implantable device of the present invention;

FIG. 5B illustrates a perspective front view of the implantable device of FIG. 5A;

FIG. 5C shows the implantable device of FIG. 5A in situ;

FIG. 5D illustrates a compression element of the implantable device of FIG. 5A;

FIG. 6A illustrates a perspective side view of another exemplary embodiment of an implantable device of the present invention;

FIG. 6B illustrates a perspective top view of the implantable device of FIG. 6A;

FIGS. 6C and 6D show a method of using the implantable device of FIG. 6A;

FIG. 7A illustrates a perspective view of another exemplary embodiment of an implantable device of the present invention;

FIG. 7B illustrates a perspective view of still another exemplary embodiment of an implantable device of the present invention;

FIG. 8A illustrates a perspective view of yet another exemplary embodiment of an implantable device of the present invention in an unexpanded state;

FIG. 8B illustrates a perspective view of the implantable device of FIG. 8A in an expanded state;

FIG. 9A illustrates a perspective view of even still another exemplary embodiment of an implantable device of the present invention;

FIG. 9B shows a plurality of implantable devices of FIG. 9A in use together;

FIG. 10A illustrates a perspective view of another exemplary embodiment of an implantable device of the present invention in an unassembled state;

FIG. 10B shows the implantable device of FIG. 10A in an assembled state;

FIG. 11A illustrates a perspective view of an exemplary embodiment of an implantable device of the present invention;

FIG. 11B shows a partial cutaway end view of the implantable device of FIG. 11A;

FIG. 11C shows another perspective end view of the implantable device of FIG. 11A;

FIG. 12 illustrates a perspective view of still another exemplary embodiment of an implantable device of the present invention;

FIG. 13 illustrates a method of using another exemplary embodiment of an implantable device of the present invention; and

FIG. 14 illustrates a perspective view of yet another exemplary embodiment of an imaging device of the present invention.

### DESCRIPTION OF THE EMBODIMENTS

The present disclosure provides a methodology, devices and instruments for diagnosing and treating joint pain to restore natural joint function and preserving, as much as possible, the joint's articular and cartilage surface. Treatments through the joint that violate the articular and cartilage surface often weaken the bone and have unpredictable results. Rather than focusing on treatment of pain through the joint, the embodiments diagnose and treat pain at its source in the subchondral region of a bone of a joint to relieve the pain. Applicants have discovered that pain associated with joints, especially osteoarthritic joints, can be correlated to bone defects or changes at the subchondral level rather than, for example, the severity of osteoarthritic progression or defects at the articular surface level. In particular, bone defects, such as bone marrow lesions, edema, fissures, fractures, hardened bone, etc. near the joint surface lead to a mechanical disadvantage and abnormal stress distribution in the periarticular bone, which may cause inflammation and generate pain. By altering the makeup of the periarticular bone (which may or may not be sclerotic) in relation to the surrounding region, it is possible to change the structural integrity of the affected bone and restore normal healing function, thus leading to a resolution of the inflammation surrounding the defect.

Applicants have discovered that treatment of the bone by mechanical and biological means to restore the normal physiologic stress distribution, and restore the healing balance of the bone tissue at the subchondral level, is a more effective way of treating pain than conventional techniques. That is, treatment can be effectively achieved by mechanically strengthening or stabilizing the defect, and biologically initiating or stimulating a healing response to the defect. Accordingly, the present disclosure provides methods, devices, and systems for a subchondral procedure. This procedure and its associated devices, instruments, etc. are also marketed under the registered trademark name of SUBCHONDROPLASTY™. The SUBCHONDROPLASTY™ procedure is a response to a desire for an alternative to patients facing partial or total knee replacement.

In general, the SUBCHONDROPLASTY™ or SCP™ technique is intended to both strengthen the bone and stimulate the bone. In SCP™, bone fractures or non-unions are stabilized, integrated or healed, which results in reduction of a bone defect, such as a bone marrow lesion or edema. In addition, SCP™ restores or alters the distribution of forces in a joint to thereby relieve pain. SCP™ can be performed arthroscopically or percutaneously to treat pain by stabilizing chronic stress fracture, resolving any chronic bone marrow lesion or edema, and preserving, as much as possible, the articular surfaces of the joint. SUBCHONDROPLASTY™ generally comprises evaluating a joint, for example, by taking an image of the joint, detecting the presence of one or more subchondral defects, diagnosing which of these subchondral defects is the source of pain, and determining an extent of treatment for the subchondral defect. The present technique is particularly suited for treating chronic defects or injuries, where the patient's natural healing response has not resolved the defect. It should be noted, however, that the technique is equally applicable to treatment of defects in the subchondral region of bone where the defect is due to an acute injury or from other violations. The present disclosure provides several exemplary treatment modalities for SCP™ for the different extents of treatment needed. Accordingly, a medical practitioner may elect to use the techniques and devices described herein to subchondrally treat any number of bone defects as he deems appropriate.

In some embodiments, detection and identification of the relevant bone marrow lesion or bone marrow edema (BML or BME) can be achieved by imaging, e.g., magnetic resonance imaging (MRI), X-ray, manual palpation, chemical or biological assay, and the like. A T1-weighted MRI can be used to detect sclerotic bone, for example. Another example is that a T2-weighted MRI can be used to detect lesions, edemas, and cysts. X-ray imaging may be suitable for early-stage as well as end-stage arthritis. From the imaging, certain defects may be identified as the source of pain. In general, defects that are associated with chronic injury and chronic deficit of healing are differentiated from defects that result, e.g., from diminished bone density. SCP™ treatments are appropriate for a BML or BME that may be characterized as a bone defect that is chronically unable to heal (or remodel) itself, which may cause a non-union of the bone, stress or insufficiency fractures, and perceptible pain. Factors considered may include, among other things, the nature of the defect, size of the defect, location of the defect, etc. For example, bone defects at the edge near the articular surface or periphery of a joint may be often considered eligible for treatment due to edge-loading effects as well as the likelihood of bone hardening at these locations. A bone defect caused by an acute injury would generally be able to heal itself through the patient's own natural healing process. However, in such situations where the bone defect is due to an acute injury and either the defect does not heal on its own, or the medical practitioner decides that the present technique is appropriate, SCP™ treatments can be administered on acute stress fractures, BML or BME, or other subchondral defects, as previously mentioned.

According to the embodiments, the SCP™ treatment may continue after surgery. In particular, the patient may be monitored for a change in pain scores, or positive change in function. For example, patients are also checked to see when they are able to perform full weight-bearing activity and when they can return to normal activity. Of note, if needed, the SCP™ procedure can be completely reversed in the event that a patient requires or desires a joint replacement or other type of procedure. The SCP™ treatment may also be performed in conjunction with other procedures, such as cartilage resurfacing, regeneration or replacement, if desired.

The present disclosure provides a number of treatment modalities, and associated devices, instruments and related methods of use for performing SUBCHONDROPLASTY™. These treatment modalities may be used alone or in combination.

In one treatment modality, the subchondral bone in the region of the bone marrow lesion or defect can be strengthened by introduction of a hardening material, such as a bone substitute, at the site. The bone substitute may be an injectable calcium phosphate ensconced in an optimized carrier material. In SCP™, the injected material may also serve as a bone stimulator that reinvigorates the desired acute bone healing activity.

For example, polymethylmethacrylate (PMMA) or calcium phosphate (CaP) cement injections can be made at the defect site. PMMA injection may increase the mechanical strength of the bone, allowing it to withstand greater mechanical stresses. CaP cement injection may also increase the mechanical strength of the bone, while also stimulating the localized region for bone fracture repair. In one embodiment, the injection can be made parallel to the joint surface. In another embodiment, the injection can be made at an angle to the joint surface. In yet another embodiment, the injection can be made below a bone marrow lesion.

In another treatment modality, the subchondral bone region can be stimulated to trigger or improve the body's natural healing process. For example, in one embodiment of this treatment modality, one or more small holes may be drilled at the region of the defect to increase stimulation (e.g., blood flow, cellular turnover, etc.) and initiate a healing response leading to bone repair. In another embodiment, after holes are drilled an osteogenic, osteoinductive, or osteoconductive agent may be introduced to the site. Bone graft material, for example, may be used to fill the hole. This treatment modality may create a better load-supporting environment leading to long term healing. Electrical or heat stimulation may also be employed to stimulate the healing process of a chronically injured bone. Chemical, biochemical and/or biological stimulation may also be employed in SCP™. For instance, stimulation of bone tissue in SCP™ may be enhanced via the use of cytokines and other cell signaling agents to trigger osteogenesis, chondrogenesis, and/or angiogenesis to perhaps reverse progression of osteoarthritis.

In yet another treatment modality, an implantable device may be implanted into the subchondral bone to provide mechanical support to the damaged or affected bone region, such as where an insufficiency fracture or stress fracture has occurred. The implant may help create a better load distribution in the subchondral region. In the knees, the implant may support tibio-femoral compressive loads. In addition, the implant may mechanically integrate sclerotic bone with the surrounding healthy bone tissue. The implant may be placed in cancellous bone, through sclerotic bone, or under sclerotic bone at the affected bone region. The implant may also be configured as a bi-cortical bone implant. In one embodiment, one side of the implant can be anchored to the peripheral cortex to create a cantilever beam support (i.e., a portion of the implant is inserted into bone but the second end stays outside or near the outer surface of the bone). The implant may be inserted using a guide wire. In one example, the implant may be inserted over a guide wire. In another example, the implant may be delivered through a guide instrument. Exemplary guide instruments, navigation, and targeting systems are also disclosed in co-pending and co-owned U.S. patent application Ser. No. 12/950,230, filed Nov. 19, 2010 and entitled “INSTRUMENTS FOR TARGETING A JOINT DEFECT,” U.S. patent application Ser. No. 12/950,154, filed Nov. 19, 2010 and entitled “INSTRUMENTS FOR VARIABLE ANGLE APPROACH TO A JOINT,” U.S. patent application Ser. No. 12/950,114, filed Nov. 19, 2010 and entitled “COORDINATE MAPPING SYSTEM FOR JOINT TREATMENT,” U.S. patent application Ser. No. 12/950,061, filed Nov. 19, 2010 and entitled “NAVIGATION AND POSITIONING INSTRUMENTS FOR JOINT REPAIR,” the contents of which are herein incorporated in their entirety by reference.

The implant may further be augmented with a PMMA or CaP cement injection, other biologic agent, or an osteoconductive, osteoinductive and/or osteogenic agent. The augmentation material may be introduced through the implant, around the implant, and/or apart from the implant but at the affected bone region, such as into the lower region of a bone marrow lesion or below the lesion. For example, the implant may act as a portal to inject the augmentation material into the subchondral bone region.

While each of the above-mentioned treatment modalities may be administered independent of one another, it is contemplated that any combination of these modalities may be applied together and in any order so desired, depending on the severity or stage of development of the bone defect(s). Accordingly, the present disclosure also provides suitable implantable fixation devices for the surgical treatment of these altered bone regions or bone defects, especially at the subchondral level. Applicants have also discovered devices and instruments that can be used in combination with cements or hardening materials commonly used to repair damaged bone by their introduction into or near the site of damage, either to create a binding agent, cellular scaffold or mechanical scaffold for immobilization, regeneration or remodeling of the bone tissue.

The embodiments of the implant may be provided with a central opening or canal extending longitudinal along the major axis, as shown, or it may be cannulated as is common in the art. The implant may slide over a guide wire for insertion. Further, the implant may be fenestrated, with pores or channels. The pore or channels may be in fluid communication with a central opening of the implant.

As previously mentioned, the implant may further be augmented with a PMMA or CaP cement injection, other biologic agent, or an osteoconductive, osteoinductive and/or osteogenic agent like a bone graft material. The augmentation material may be introduced through the implant, around the implant, and/or apart from the implant but at the affected bone region, such as into the lower region of a bone marrow lesion. For example, the implant may act as a portal to inject the augmentation material into the bone tissue.

The present embodiment provides structural features to accommodate these scenarios. It is contemplated that an end of the implant may be configured to allow a quick release connection with a tool, such as for example a threaded connection.

The tool could be, for example, an insertion tool, an injection needle, or a catheter. In one embodiment, the implant can be inserted and twisted to lock into the tool or system. Alternatively, the implant may be provided with a Luer lock-type mechanism for attachment to an injection system.

If provided, the central opening of the implant would enable the augmentation material to be introduced through the implant, and channels around the implant would allow the material to be ejected around the implant. Pores and channels can also provide access for bone ingrowth and vasculature permeation. The pores or channels may be provided in any variety of sizes; however, it is understood that adjustment of the pore size would allow the user to control the flow of an injectable material through the implant.

By making the pores smaller, resistance to flow is increased and alternatively by making the pores larger, resistance to flow is reduced. It is therefore contemplated that the implant may be provided with suitably sized pores for use with the intended injectable material desired. For instance, the pores or channels may have a larger dimension than the central opening, creating a path of least resistance for injected material through the channels and thereby reducing backflow out of the central opening.

Alternatively, it is possible to provide the implant with channels or pores in a region of the implant's body. It is further contemplated that a plug or cap may be provided with implant in order to seal off the central opening 30 and thereby prevent any augmentation material contained within to leak out.

The implant may be formed of any suitable biocompatible material, including metal or polymer materials. Suitable metals may include, but are not limited to, stainless steel, titanium, titanium alloys, and cobalt chrome, as examples. Porous metals may also be appropriate. The implant may also be ABS injection molded plastic, polyetheretherketone (PEEK), polyethylene (PE), or ultra high molecular weight polyethylene (UHMWPE). If desired, the implant may be bioabsorbable or bioresorbable. In some embodiments, the implant may be formed of allograft or cadaver bone, including cortical, cortico-cancellous, bi-cortical, tri-cortical, or sesamoid bone material. In other embodiments, the implant may be formed partially or wholly from a radiolucent material. For example, the implant may be formed from a material blended with a radiopaque material, such as barium sulfate. In addition, radiopaque markers may be employed with the implant for imaging possibilities.

As illustrated in the following figures, the implant may be shaped so as to have varying diameters along its length. For instance, the implant may have an overall threaded configuration, a figure “8” shape, a bowling pin shape, a U-shape, a crescent or C-shape, an I-beam shape, a rectangular or square shape, a star shape, or corkscrew shape, etc. so long as it is suitable for insertion into bone tissue and has enough structural integrity to perform its intended function of bridging a fracture or fissure, supporting bone regrowth or remodeling, and/or binding the bone tissue together to prevent further breakdown or degeneration.

The implant of the present disclosure may be used to repair bone defects in a joint region such as the knee, shoulder, ankle, hip or other joint of the patient's body. The implant may be useful, for example, in repairing an insufficiency fracture of a bone at a joint. The implant may serve as a fusion device, enabling rigid fixation at the defect site. For instance, the implant may serve as a useful facet fusion device. Alternatively, the implant may be configured to facilitate the patient's natural healing process without fusion at the defect site.

If desired, the implant may also include a biological agent. The biological agent may be included in a coating on the implant. Alternatively, the biological agent may be embedded inside the implant. Suitable biological agents may include, for example, osteogenic, osteoconductive and/or osteoinductive agents. In addition, a bioactive agent such as platelet rich plasma (PRP), bone marrow aspirate (BMA), bone morphogenic protein (BMP), demineralized bone matrix (DBM), stem cells, or allograft material, for example, may also be employed. Furthermore, a bioactive surface may be created on the implant by treating the implant with, for example, acid etching, grit blasting, plasma spraying, bioactive glass coating, photo-chemical etching, or other suitable surface treatments for creating a roughened surface.

While the implant has been described as being used with an injectable material, it is understood, however, that the implant shown here, as well as the other implants and devices described herein, may be used alone without any injectable material. Turning now to the drawings, mechanical fixation devices particularly suitable for implantation in certain areas of the bone, such as the periarticular surface or the subchondral bone area (usually within the range of about 2-15 mm from the bone surface) are shown.

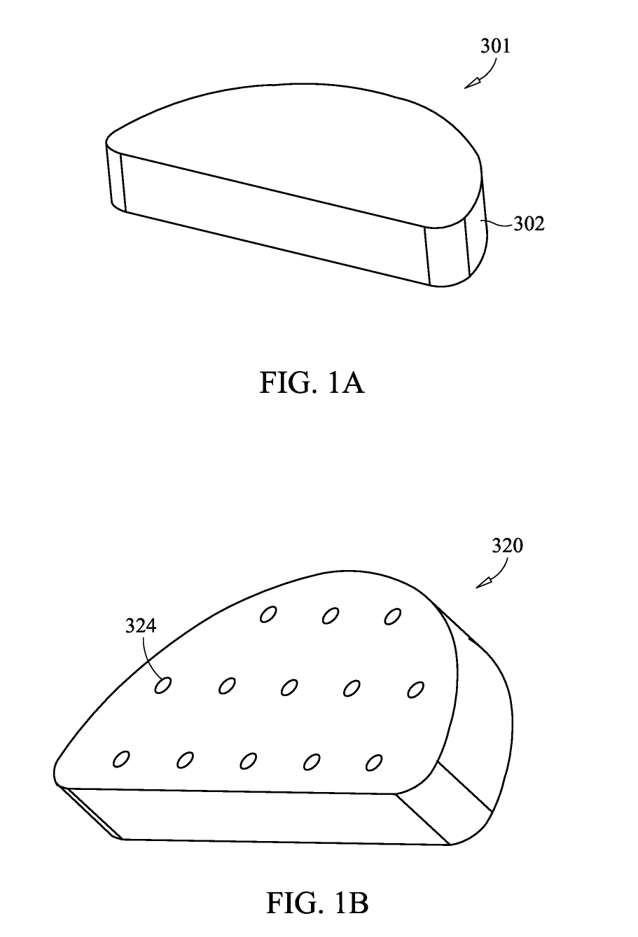

FIGS. 1A and 1B represent an exemplary embodiment of such an implantable device 300 having a disc shape. As shown in FIG. 1A, the implant 300 may have a smooth, rounded side 302 that can be placed facing the exterior of the bone so as to create an overall smooth profile once the implant 300 has been inserted. If desired, a disc-shaped implant 320 may be provided with pores 324 for tissue ingrowth, for example.

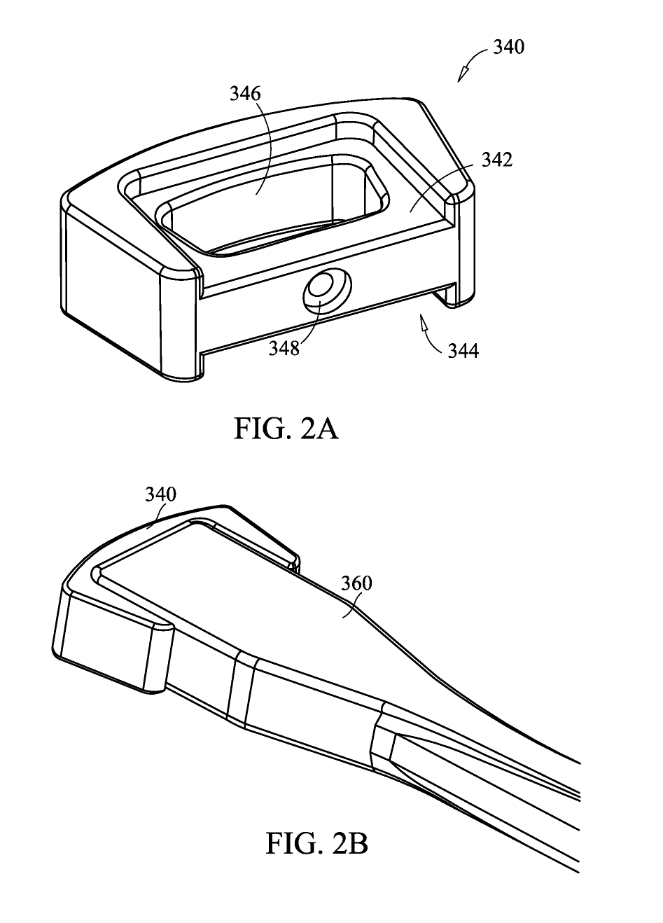

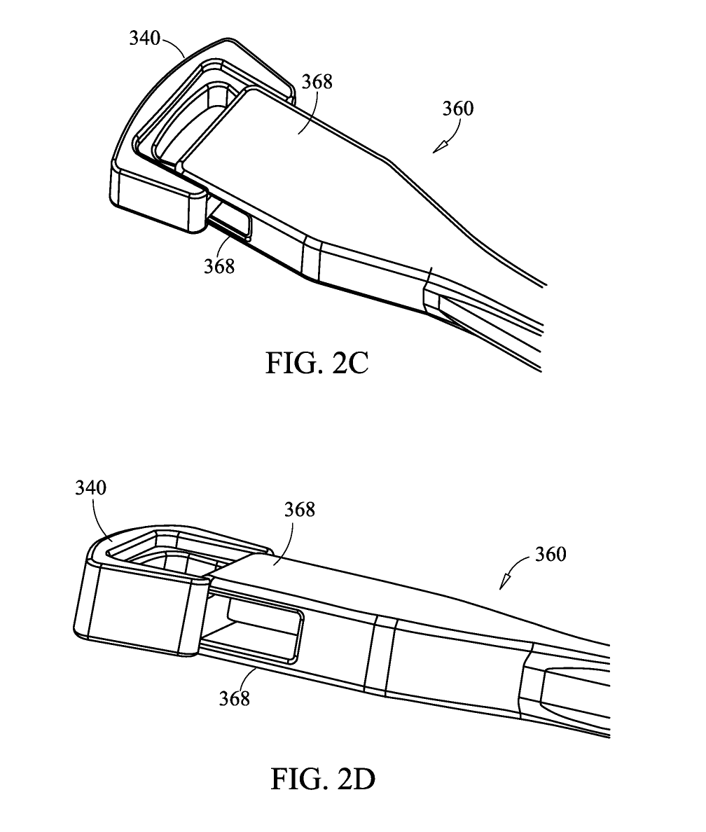

FIGS. 2A-2D represent another exemplary embodiment of an implantable device 340 having a wedge shape. The implant 340 may include a top recessed portion 342 and a bottom recessed portion 344 between which extends a central opening 346, as shown in FIG. 2A. The central opening 346 may hold bone graft material, for example. A tool-engaging opening 348 may also be provided.

Also provided may be an insertion tool 360 configured for use with the implant 340. Insertion tool 360 may include a pair of tongs 368 that is configured to seat against the top and bottom recessed portions 342, 344 of the implant 340. When fully engaged with the implant 340, the tongs 368 may serve to protect any bone graft material residing within the central opening 346 during insertion into bone. After the implant 340 has been inserted, the insertion tool 360 may be removed by sliding the tongs 348 away from the implant 340, as illustrated in FIGS. 2C and 2D. In another embodiment, it is contemplated that the insertion tool 360 may be configured with an injection portal such that a flowable material could be introduced through the tool 360 and into the implant 340. For example, the insertion tool 360 could be provided with a multi-lumen shaft that would enable the user to inject a material through the shaft and tool-engaging opening 348 into the central opening 346. The tongs 368 could act to prevent unintended seepage of the material out of the implant 340, and may be retracted during the injection process in a controlled manner, leaving just the implant 340 with the flowable material behind.

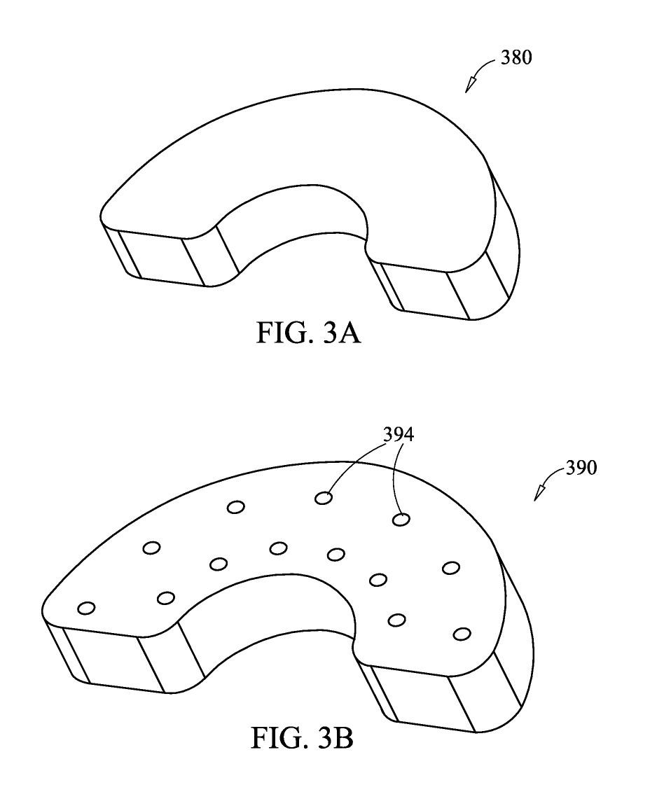

In yet another embodiment, the implantable device 380 may have a crescent or moon shape. As with implantable device 300, 320, the implantable device 380 shown in FIGS. 3A and 3B may be fashioned as a solid body, or the implantable device 390 may include pores 394.

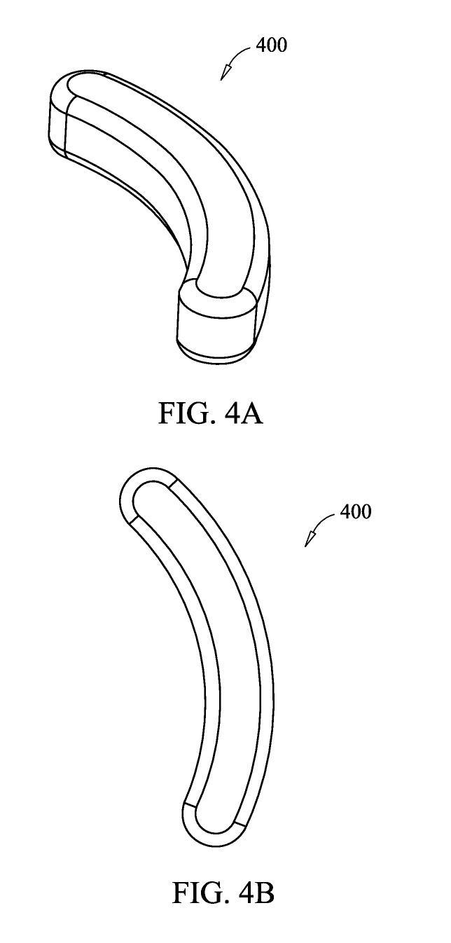

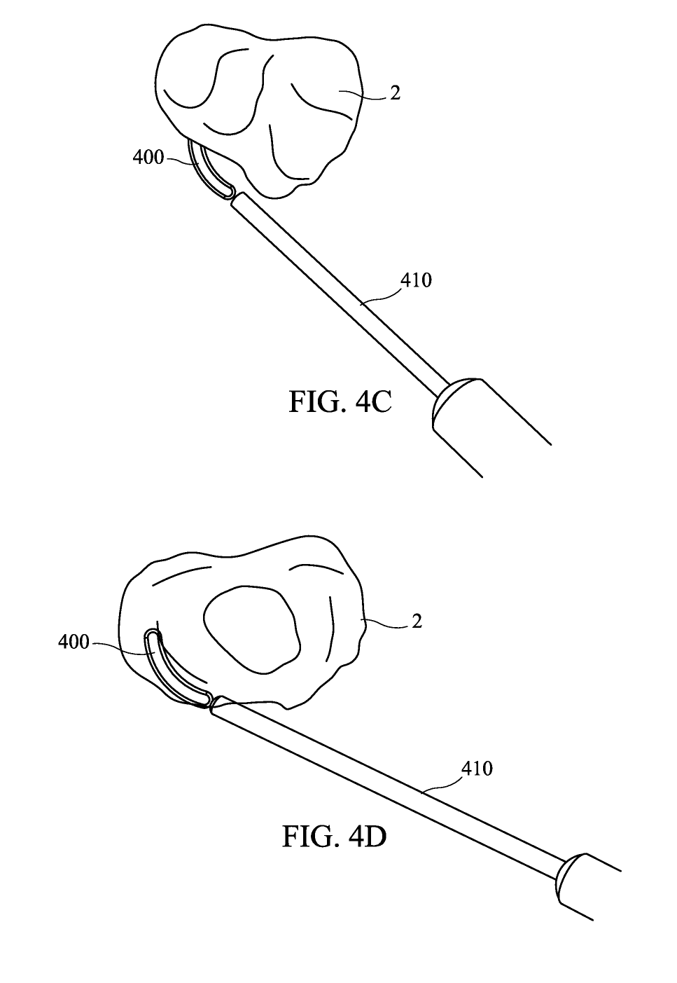

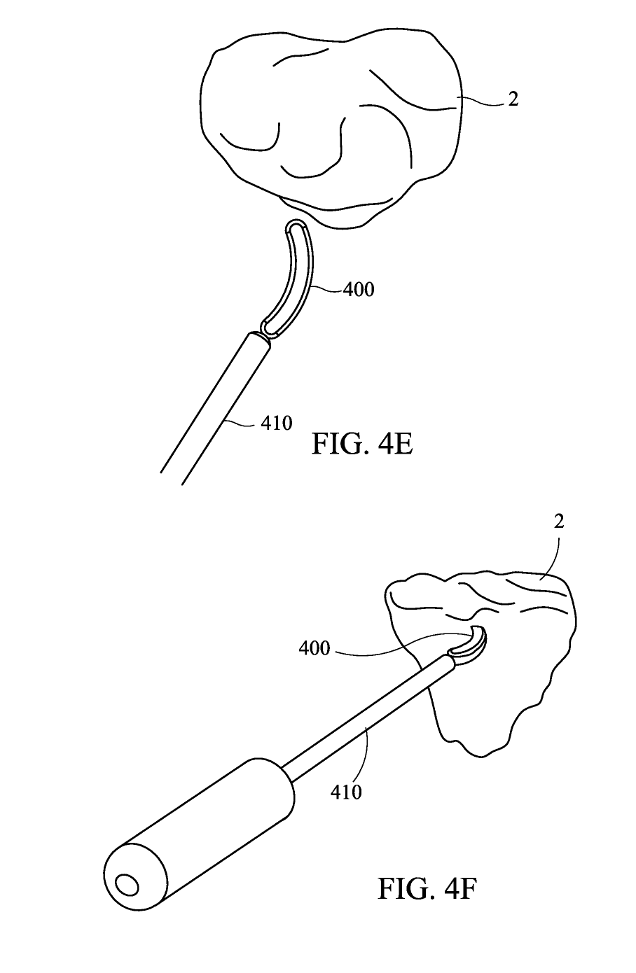

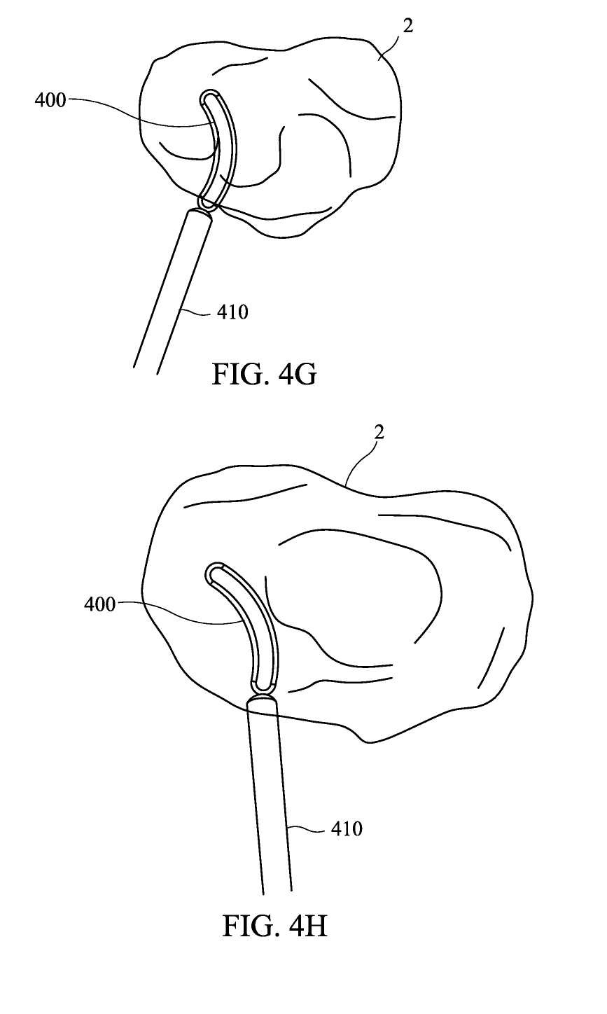

In still another embodiment, a banana shaped or curved implantable device 400 is provided. The curved implantable device 400 may be provided as a solid body, as shown in FIGS. 4A and 4B, or may be provided with pores (not shown). It is contemplated that a curved implant 400 may be desirable where the bone defect, such as a lesion, occurs near the periphery of the bone 2. For these peripheral lesions or defects, a curved implant 400 may be placed such that the implant 400 matches the contour of the bone 2 being treated. FIGS. 4C-4H illustrate exemplary methods of using a curved implant 400 of the present disclosure to treat a peripheral defect.

As shown, the curved implant 400 may be attached to an insertion tool 410 such as for example, by a threaded connection between a threaded hole (not shown) in the implant 400 and a threaded end (not shown) of the insertion tool 410. The curved implant 400 may be implanted in an open procedure, or in a minimally invasive procedure, depending on how soft the bone 2 is at the site of insertion. The implant 400 could be press-fit into the bone 2 for example.

In one example, the curved implant 400 may be inserted so that the curved surface of the implant 400 matches the curves of the bone 2 to be treated, as shown in FIG. 4D. Placement of the implant 400 in this manner spares the rest of the bone 2 from further obstacles, and enables the bone 2 to receive additional devices if so desired. For instance, in a tibial bone by maximizing the space available in the bone 2, the patient may receive a knee implant with a keel, for example, in addition to having the curved implant 400.

Of course, it is also possible to implant the curved implant 400 in a manner such that the curvature does not match the curvature of the bone 2, as shown in FIGS. 4E-4G. It is also contemplated that a plurality of curved implants 400 may be utilized together, where it may be desirable to encircle or enclose a defect in a bone 2. As shown in FIG. 4H, two or more curved implants 400 may be placed inside a bone 2 in order to encase the defect or area to be treated.

In addition, it has been discovered that the bone tissue surrounding a bone marrow lesion tends to be relative soft (usually, edema is present) compared with normal, healthy bone tissue. Accordingly, according to SCP™, the surgeon may also treat the lesion or defect by compacting the soft bone tissue and then optionally inserting an implant, such as curved implant 400, into the area adjacent to the compacted bone tissue.

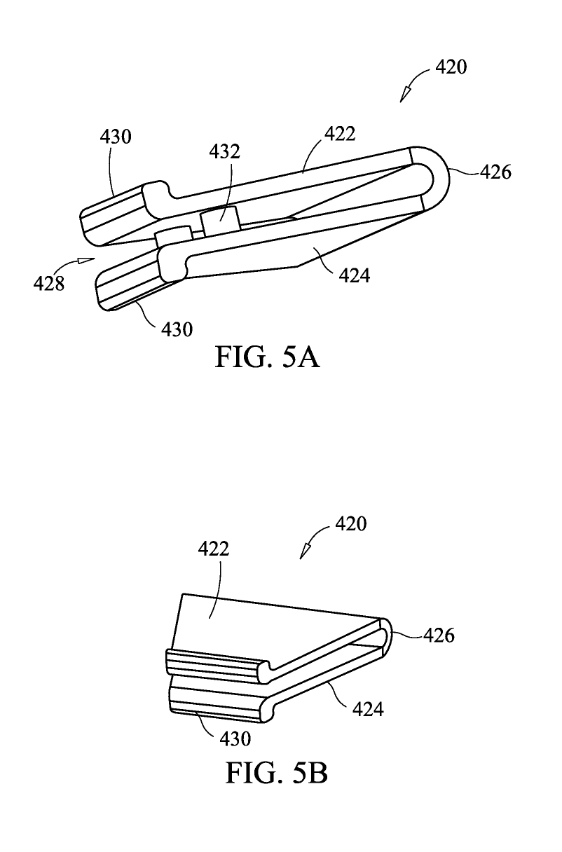

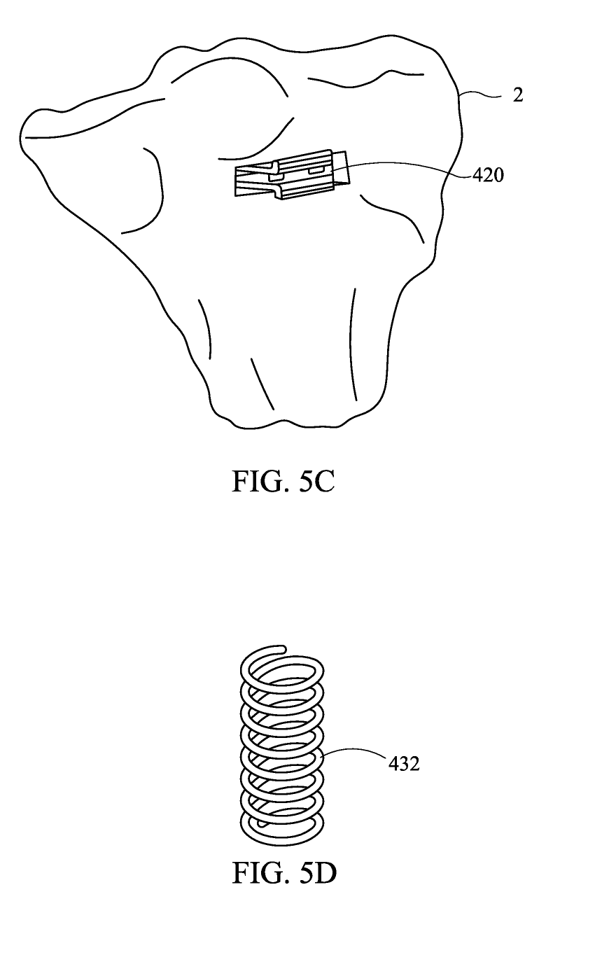

FIGS. 5A-5D illustrate an example of a resilient implant 420 of the present disclosure. Implant 420 may include a top plate 422 and a bottom plate 424 connected by a connecting wall 426. Implant 420 may include an open end 428 that may terminate into lips 430, if desired. Within the implant 420 a spring or conformable element 432, as shown in FIG. 5D, may reside. The implant 420 may have an overall wedge shape, as shown in FIG. 5B. The implant 420 may be inserted into a void where bone has been resected, as shown in FIG. 5C.

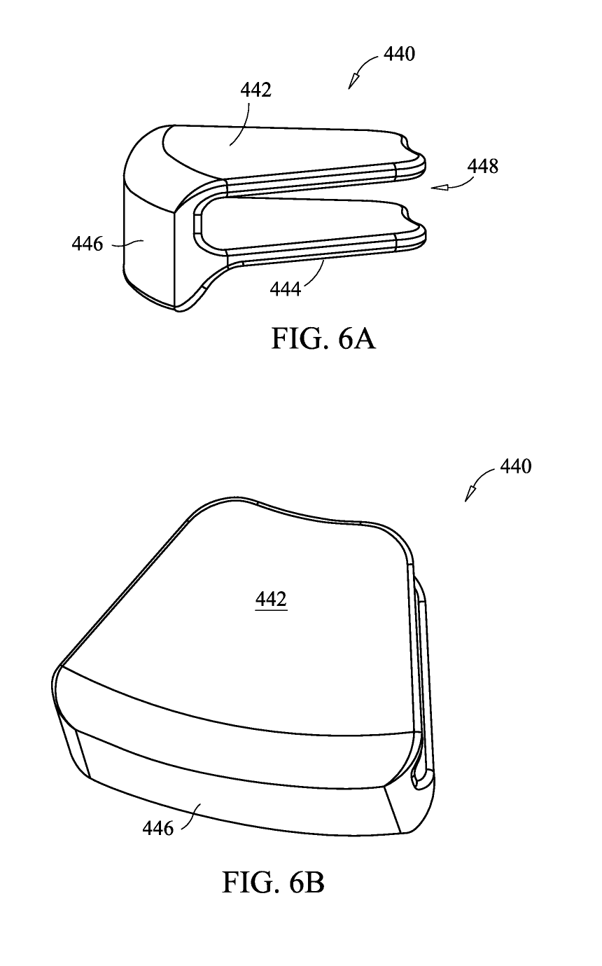

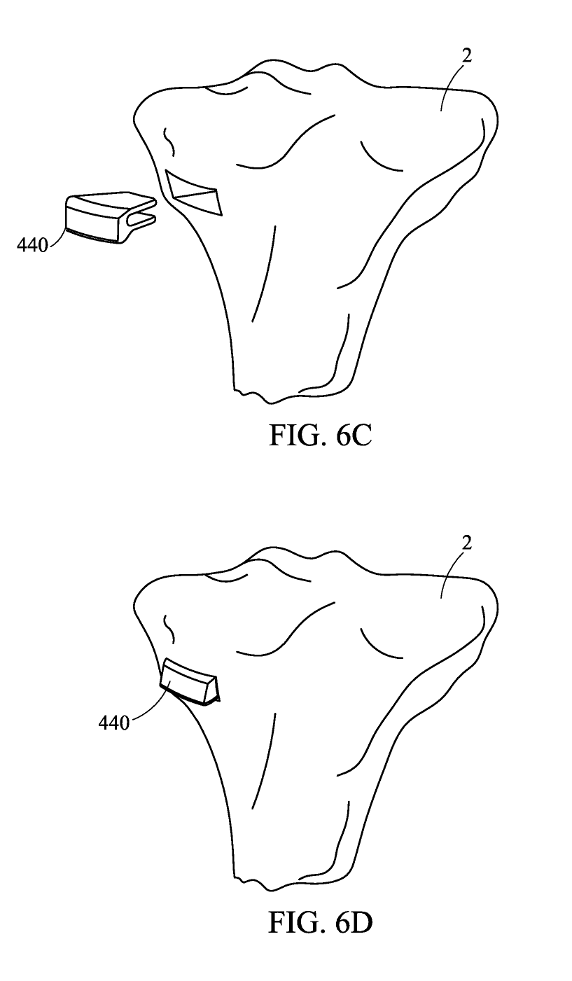

FIGS. 6A-6D illustrate yet another example of a resilient implant 440 of the present disclosure. Implant 440 may include a top plate 442 and a bottom plate 444 connected together by a connecting wall 446. As with resilient implant 420, the implant 440 may have an open end 448, which allows the implant 440 some degree of deformity or flexibility where such a property is desirable. The implant 440 may have an overall wedge shape. Further, if so desired, the connecting wall 446 may be curved so that the implant 440 matches the contours of the bone 2 to be treated and creates an overall smooth profile once implanted. In addition, the top and bottom plates 442, 444 may be configured to enable the implant 440 to be inserted in a press-fit fashion into soft bone tissue if appropriate.

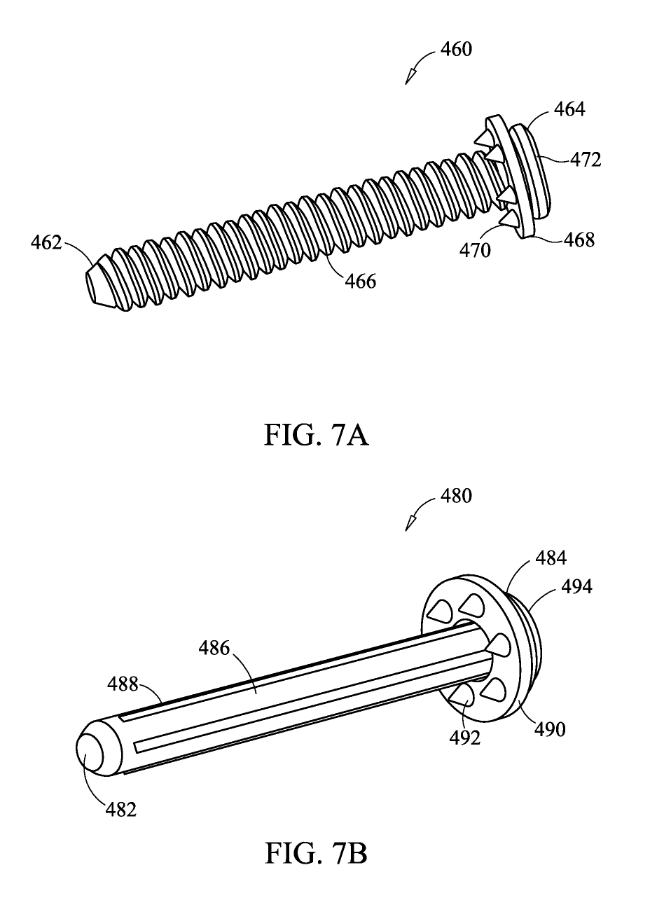

FIGS. 7A and 7B show more examples of implantable devices of the present disclosure. In FIG. 7A, a bicortical bone screw 460 is shown. The screw 460 may be of the type having a first, leading end 462 extending into a threaded shaft 466 and terminating in a second, trailing end 464. The screw 460 may be provided with a flange 468 having on its underside a surface feature for bone purchase, such as for example, spikes 470. The second, trailing end 464 may also include a cap 472. The bone screw 460 may be suitable for use with the present invention where it is desirable to have a portion of the screw 460, such as the flange 468, anchored to the outside of the bone being treated.

Likewise, FIG. 7B provides a pin 480 that may be anchored to an outer surface of a bone to be treated. Pin 480 may have a first, leading end 482, an elongate body 486, and a second, trailing end 484 with a cap 494. A flange 490 may be provided with a surface feature on its underside, such as for example, spikes 492 as shown. The spikes 492 enable the flange to anchor to the bone surface. Fins 488 or other surface features may be provided on the elongate body 486 to allow the pin 480 to attach to bone tissue. It is contemplated that other shapes may be employed for the pin, such as a T- or L-shape to enable the device to not only support the weakened area around the bone defect, but also additionally anchor to the outer surface of the bone.

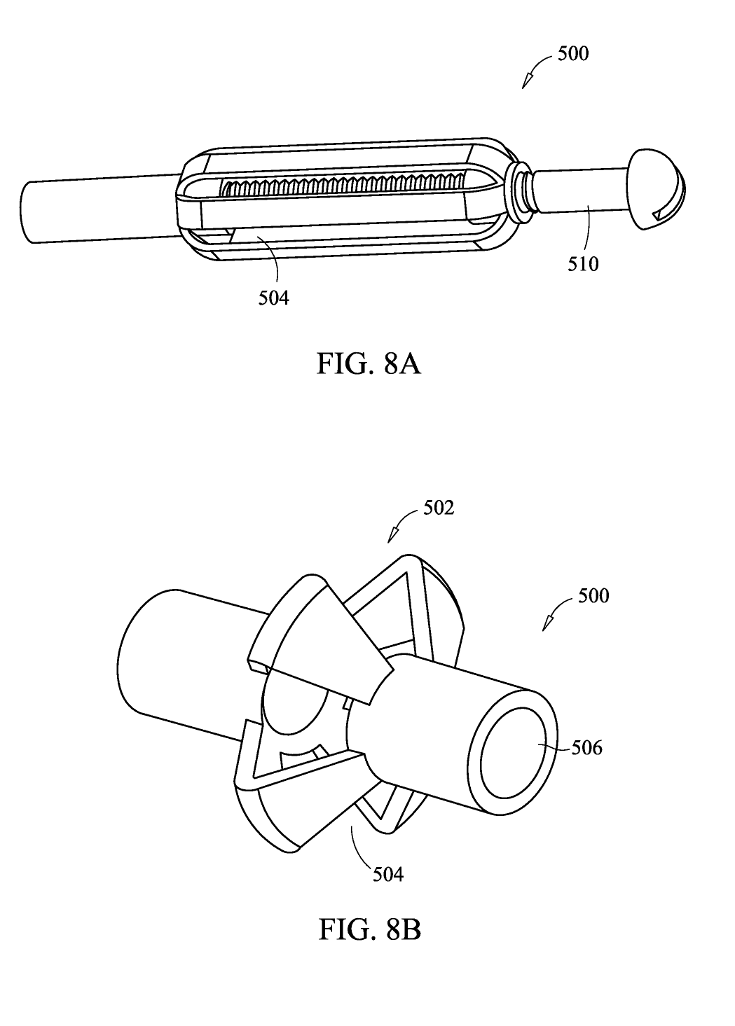

In another embodiment of the present disclosure, an expandable device 500 is provided. FIGS. 8A and 8B show an expandable device 500 having a central body 502 with a plurality of slots 504 that can be collapsed to expand the central body 502 as shown in FIG. 8B. A central threaded opening 506 may be provided to receive a threaded screw 510 for effecting the expansion. If so desired, bone graft material may be placed inside the central body 502 to enhance bone growth.

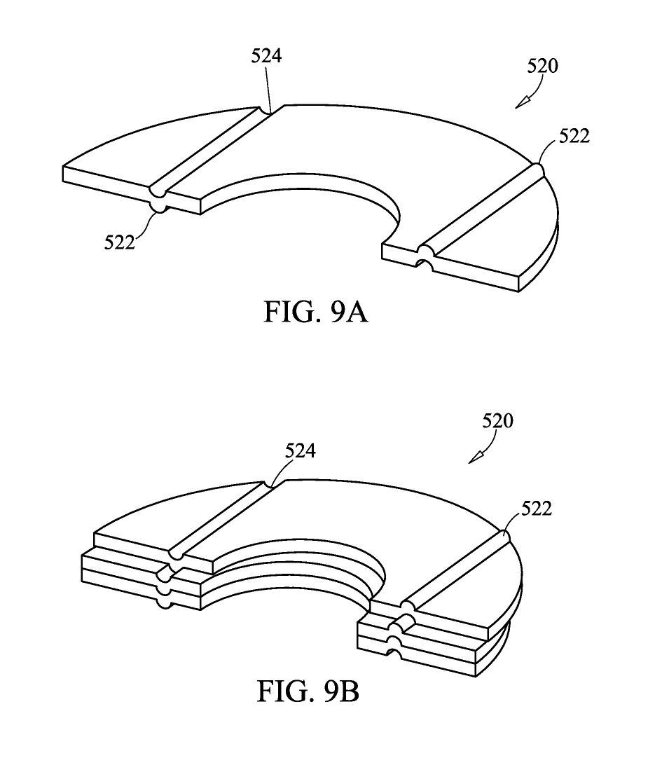

FIGS. 9A and 9B illustrate another embodiment of the present disclosure. As shown, an implant 520 may be provided with ridges or protrusions 522 and corresponding grooves or notches 524 on its outer surface. The ridges 522 and grooves 524 serve to interlock implants 520 together as shown in FIG. 9B. Accordingly, the user may be able to build a suitable implant for a resected bone segment by stacking or layering a plurality of implants 520 together.

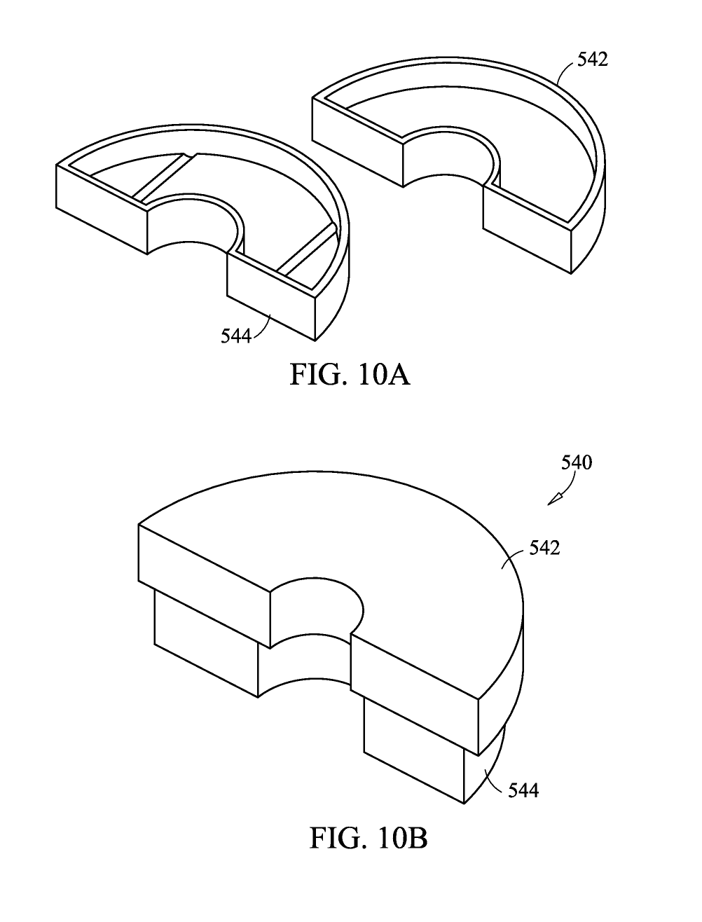

FIGS. 10A and 10B show an implant 540 of the present disclosure comprising a pair of top and bottom shells 542, 544 which can be placed together to form a container that can receive bone graft material, for example. The shells 542, 544 may be porous so as to facilitate bone growth therethrough. In one embodiment, the assembled implant 540 could be expandable to fit the defect or insertion site once in place.

Although not shown, in one embodiment, the implantable device may be a 3-dimensional envelope or pouch implantable in a first, smaller configuration and deployable to a second, larger or full configuration after it is in place. The device may be filled with an osteogenic, osteoconductive, and/or osteoinductive material such as those mentioned above. In one example, the device may be filled with a bone cement such as PMMA. In another example, the device may be filled with bone graft material.

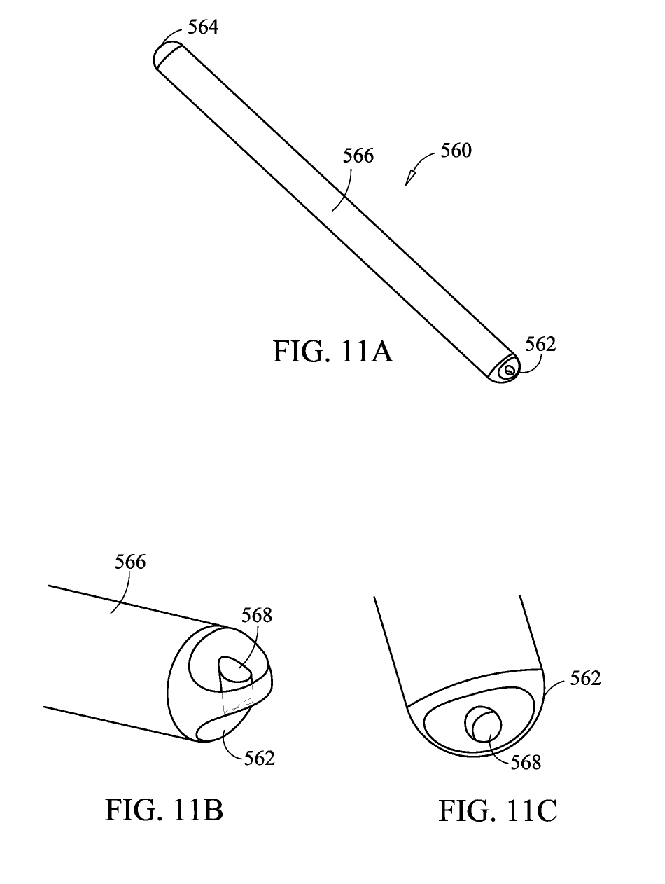

FIGS. 11A-11C illustrate still another embodiment of the present disclosure, whereby a rod shaped implant 560 is provided. The implant may include a first, leading end 562, a second, trailing end 564 and an elongate body 566 extending therebetween. The first, leading end 562 may be tapered if desired, and may additionally include a hole 568 to receive a fixation device, such as for example, a suture or pin (not shown).

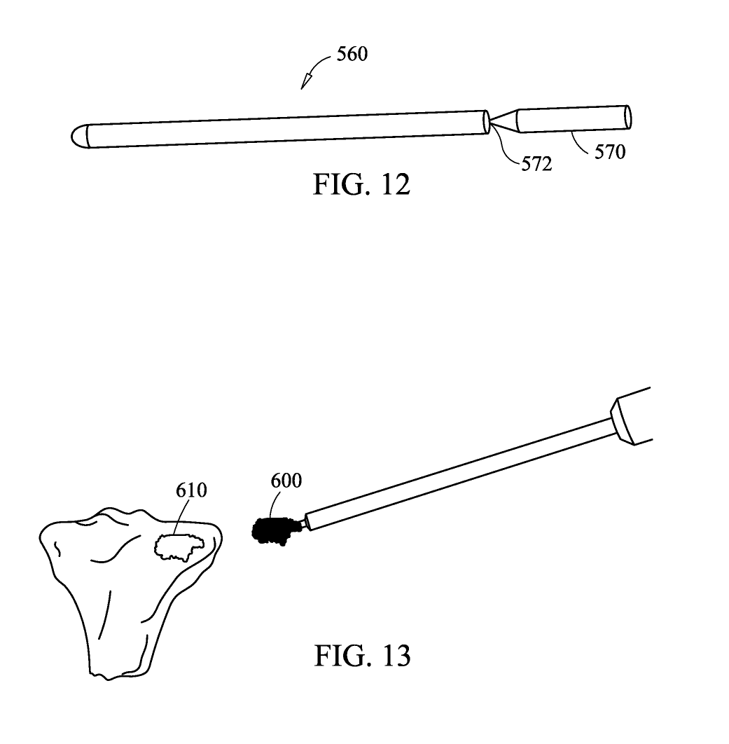

FIG. 12 shows a variation of the rod shaped implant 560 whereby the implant 560 includes a breakaway portion 570. Accordingly, the user may secure the implant 560 entirely within bone and leave the breakaway portion 570 outside the bone. When the implant 560 has been properly secured, the breakaway portion 570 may be snapped off at the break point 572 (usually a scored or thinned section) to leave the implant 560 flush with the bone surface.

Devices of the present disclosure may be formed in situ or outside the patient and later implanted. The device may be non-uniform or asymmetric in shape. In one example, the device may be formed of a plurality of similar or different subcomponents, for example, a linked chain of balls containing biologic agents. In some embodiments, the device may be customized to the patient. For example, as shown in FIG. 13, using 3-dimensional imaging technology, it may be desirable to provide an equally 3-dimensional implant 600 that matches precisely the anatomical site 610 where the implant 600 is to be placed. This would ensure conformability and avoid a less than perfect match between the implant and the implantation site.

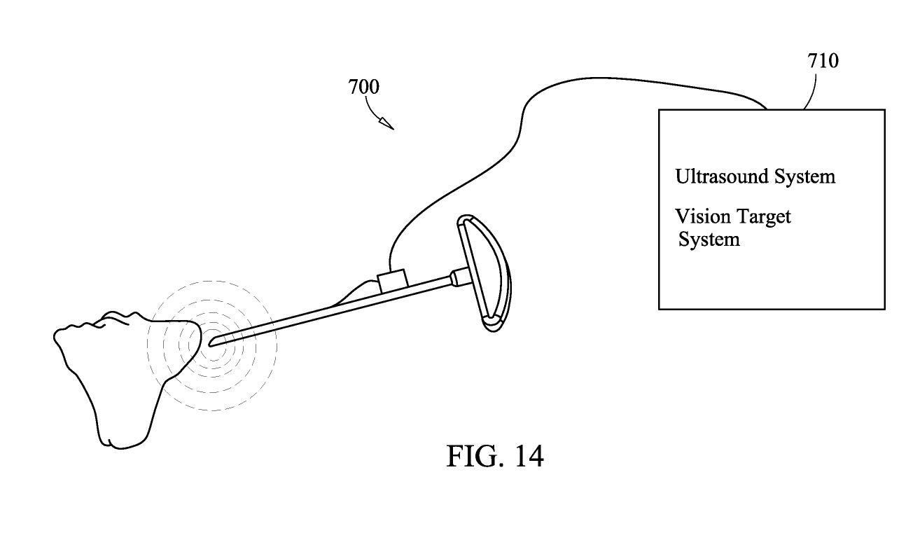

In addition, it is contemplated that any of the devices described in the present disclosure may be used in conjunction with an imaging tool 710 in a system 700 as shown in FIG. 14 that would allow the user the benefit of visualizing the lesion site, either before, during or after insertion into the bone to be treated.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

What is claimed is:

1. An implantable device for insertion into a periphery of a bone, comprising:
a curved elongate body extending between a first, leading end and a second, trailing end, the second end including a tool-receiving portion for receiving a tool.

2. The device of claim 1, wherein the tool-receiving portion comprises a threaded bore.

3. The device of claim 1, wherein at least a portion of the curved elongate body is smooth.

4. The device of claim 1, wherein the first end is tapered.

5. The device of claim 1, wherein the curved elongate body comprises at least one surface feature to enhance bone attachment.

6. The device of claim 5, wherein the at least one surface feature comprises at least one spike, barb, or tooth.

7. A method of treating a bone defect near a periphery of the bone, comprising the steps of:
providing a curved implantable device having a tool-engaging feature;
securing the device to an insertion tool; and
using the insertion tool, inserting the curved implantable device along the periphery of the bone.

8. The method of claim 7, wherein inserting the curved implantable device comprises inserting the curved implantable device in a subchondral region of a bone.

9. The method of claim 7, wherein inserting the curved implantable device comprises inserting the curved implantable device along a subchondral access path to a subchondral defect that preserves an articular surface of a bone.

10. The method of claim 7, further comprising drilling the access path to the subchondral defect.

11. The method of claim 7, further comprising compacting bone tissue prior to inserting the curved implantable device.

12. The method of claim 7, further comprising injecting a bone hardening material via the subchondral access path.

13. A method of treating a bone defect, comprising the steps of:
providing a first implantable device, the device having a curved shape forming a concave inner surface;
providing a second implantable device, the device having a curved shape forming a concave inner surface; and
inserting the first and second implantable devices such that the concave inner surfaces face towards one another and encircle the bone defect.

14. The method of claim 13, wherein inserting the first and second implantable devices comprises inserting the first and second implantable devices such that the concave inner surfaces face towards one another and encircle a subchondral bone defect.

15. The method of claim 13, wherein inserting the first and second implantable devices comprises inserting the first and second implantable devices along a subchondral access path to a subchondral defect that preserves an articular surface of a bone.

16. The method of claim 13, further comprising drilling the access path to the subchondral defect.

17. The method of claim 13, further comprising compacting bone tissue prior to inserting the curved first and second implantable device.

18. The method of claim 13, further comprising injecting a bone hardening material via the subchondral access path.
